(19) United States
(12) Patent Application Publication
(10) Pub. No.: US 2009/0306639 A1
(43) Pub. Date: Dec. 10, 2009

(54) Cryoprobe incorporating electronic module, and system utilizing same

(75) Inventors: Yoav Nevo, Kochav Yair (IL); Gil Ofir, Even-Yehuda (IL)

Correspondence Address: MARTIN D. MOYNIHAN d/b/a PRTSI, INC., P.O. BOX 16446, ARLINGTON, VA, 22215, US

(73) Assignee: Galil Medical Ltd., Yokneam (IL)

(21) Appl. No.: 12/457,338

(22) Filed: Jun. 8, 2009

Related U.S. Application Data
(60) Provisional application No. 61/129,153, filed on Jun. 6, 2008.

(51) Int. Cl. A61B 18/02; G06Q 30/00; G06Q 50/00

(52) U.S. Cl. 606/21; 705/2

(57) ABSTRACT

A cryotherapy system comprises a cryoprobe comprising, for example, either an electronic module including a memory or a response module operable to respond to a query signal with a response signal. A communications interface uses these modules to establish a unique identification of the cryoprobe, and a control module regulates delivery of cryogen to the cryoprobe according to calculations at least partially based on stored data associated with that unique cryoprobe identification.

### RELATED APPLICATION

This application claims the benefit of priority of U.S. Provisional Patent Application No. 61/129,153, filed on Jun. 6, 2008, the contents of which are incorporated herein by reference.

### FIELD AND BACKGROUND OF THE INVENTION

The present invention, in some embodiments thereof, relates to cryoprobes and systems utilizing cryoprobes.

Cryoprobes and cryoprobe systems according to prior art typically comprise one or more cryoprobes connectable to a cryogen supply module which comprises a cryogen source and a controller. The controller is typically designed to receive control commands from a surgeon or other operator and, following those commands, to control valves governing delivery of cryogen from the cryogen source to the connected probes. In this manner a surgeon, by commanding actions of the controller, controls delivery of cryogen to the cryoprobes, thereby controlling cooling and optionally heating of those probes.

Cryoprobes comprise cooling modules, most often powered by expansion of a high-pressure gas such as argon, or by evaporation of a liquefied gas. These cooling modules are usually operable to cool the probes to cryoablation temperatures. Cryoprobes often also comprise heating capabilities, typically supplied either by expansion of a high-pressure heating gas such as helium or by electrical resistance heating. Cryoprobes may also comprise thermal sensors operable to report temperatures within or without the probes to the system controller, such as thermocouples or thermistors, or electrical heating elements whose temperature may be calculated as a function of current flow therethrough.

Cryoablation systems comprising cryoprobes, cryogen sources and a cryogen supply controller may also comprise additional surgical probes used in conjunction with cryoprobes, such as independently insertable heating probes and independently insertable sensor probes comprising one or more thermal sensors.

Cryoprobes have been supplied in a kit designed for use in a single surgical procedure, each kit comprising a set of probes, usually the maximum number likely to be needed for an anticipated procedure. The probes are supplied in sterile packaging and accompanied by an activation key. The activation keys in the form of a “smart card” comprising a disposable one-time code which is required by the system controller before activation of the cryosurgery system can proceed.

### SUMMARY OF THE INVENTION

The present invention, in some embodiments thereof, relates to a cryoprobe having a treatment head operable to be cooled to cryoablation temperatures, the cryoprobe comprising an electronic module which includes a memory element. In some embodiments according to the invention a cryoablation system comprises one or more such cryoprobes, a cryogen supply, and a controller operable to interact with the electronic module(s) of the cryoprobes and further operable to control delivery of cryogen from the cryogen supply to the cryoprobe(s). In some embodiments the controller is programmed to read data from the electronic module memory (or memories) and to calculate and execute commands controlling flow of cryogen and/or heating gas and/or electric power for heating or other purposes to the cryoprobe, the calculations being at least partially based on data read from memories embedded in one or more cryoprobes.

The present invention, in some additional embodiments thereof, relates to a cryoprobe having a treatment head operable to be cooled to cryoablation temperatures, the cryoprobe comprising a response module operable to receive a query signal from a controller and to send a response signal in response to said query signal. In some embodiments a cryoablation system comprises one or more such cryoprobes, a cryogen supply, and a controller operable to send a query signal to the response module(s) of the cryoprobes and to receive a response signal therefrom, and further operable to control delivery of cryogen from the cryogen supply to the cryoprobe(s). The controller comprises an inquiry mechanism operable to send the inquiry signal to the cryoprobe and is operable to uniquely identify the cryoprobe upon receipt of a response signal sent by the cryoprobe in answer to said inquiry signal. The controller further comprises a memory for recording information about uniquely identified cryoprobes, a cryogen flow control mechanism for regulating flow of cryogen from the cryogen supply to the cryoprobe; and a calculation module for calculating cryogen flow commands which influence operation of the cryogen flow control mechanism, the calculation being based at least in part on information associated with the uniquely identified cryoprobe and stored in the memory. Optionally, the controller memory may be physically distant from the controller, e.g. accessed through a network or through the internet.

According to an aspect of some embodiments of the present invention there is provided a cryotherapy system comprising
a) at least one cryoprobe which comprises
   i) a treatment head coolable by delivery thereto of a cryogen; and
   ii) a response module operable to receive a query signal from a controller and to send a response signal in response to the query signal;
b) a cryogen supply; and
c) a cryogen control module which comprises
   i) an inquiry mechanism operable to send an inquiry signal to the cryoprobe and to uniquely identify the cryoprobe upon receipt of a response signal sent by the cryoprobe in answer to the inquiry signal;
   ii) a first memory for recording information about uniquely identified cryoprobes;
   iii) a cryogen flow control mechanism for regulating flow of cryogen from the cryogen supply to the cryoprobe; and
   iv) a first calculation module for calculating cryogen flow commands which influence operation of the cryogen flow control mechanism, the calculation being based at least in part on information associated with the uniquely identified cryoprobe and stored in the first memory.

According to some embodiments of the invention the response module comprises a second calculator operable to calculate the response signal as a mathematical function of a value presented by the inquiry signal.

According to some embodiments of the invention the response module is operable to recognize when a received inquiry code possesses a predetermined characteristic, and to emit a characteristic response when an inquiry code having the predetermined characteristic is recognized.

According to some embodiments of the invention the predetermined characteristic is a digital code uniquely associated with the cryoprobe.

According to some embodiments of the invention the inquiry signal is sent when an electronic communications pathway is first established between the controller and the cryoprobe.

According to some embodiments of the invention the system further comprises an information source physically distinct from the controller and from the cryoprobe, readable by the controller and comprising information characterizing the cryoprobe.

According to some embodiments of the invention the information source is a second memory device which is portable.

According to some embodiments of the invention the information source is input to the controller over an internet connection.

According to some embodiments of the invention the second memory device comprises a recordable magnetic strip.

According to some embodiments of the invention the second memory device comprises an optically readable code.

According to some embodiments of the invention the controller is programmed to record results of operational testing of the cryoprobe.

According to some embodiments of the invention the controller is operable to record information attesting to the cryoprobe having undergone operational testing, and to prevent clinical use of the cryoprobe if such information has not been so recorded.

According to some embodiments of the invention the controller is programmed to record events of usage of the cryoprobe, and to prevent supply of cryogen to the cryoprobe if more than a predetermined amount of usage has been recorded.

According to some embodiments of the invention the information characterizing the cryoprobe comprises manufacturing specifications describing the cryoprobe.

According to some embodiments of the invention the controller is operable to receive and record sensor values detected during testing of the cryoprobe, and is further operable to calculate cryogen supply parameters for use during operation of the cryoprobe as a function of the recorded values.

According to an aspect of some embodiments of the present invention there is provided a method for cryosurgery, comprising
a) reading information descriptive of a cryoprobe into a controller;
b) using the controller to associate the read information with a cryoprobe by
   i) sending an inquiry signal based on the read information to a cryoprobe,
   ii) receiving a response signal from the cryoprobe in response to the inquiry signal; and
   iii) associating the read information with the cryoprobe if and only if the response signal conforms to predetermined criteria; and
c) using the controller to calculate commands controlling supply of cryogen to the cryoprobe, the calculation being at least partially based on read information which the controller has associated with the cryoprobe in response to the response signal.

According to some embodiments of the invention the read information comprises a code which, when sent to the cryoprobe in an inquiry signal, will provoke a response signal which uniquely identifies the cryoprobe.

According to some embodiments of the invention the read information comprises at least one of a group consisting of
a) information characterizing a usage history of the cryoprobe;
b) data derived from an operational test of the cryoprobe;
c) a type designation for the cryoprobe; and
d) a descriptive characterization of the cryoprobe.

According to an aspect of some embodiments of the present invention there is provided a method for regulating use of a cryoprobe, comprising:
a) reading information descriptive of a cryoprobe into a controller;
b) associating the read information with a cryoprobe attached to a controller by
   i) sending an inquiry signal based on the read information to a cryoprobe,
   ii) receiving a response signal from the cryoprobe in response to the inquiry signal; and
   iii) associating the read information with the cryoprobe and with a unique identity tag if and only if the response signal conforms to predetermined criteria;
c) recording an activity history of the cryoprobe by recording probe usage events related to use of the cryoprobe in a memory record associated with the unique identity tag; and
c) regulating use of the cryoprobe by controlling cryogen flow as a function of recorded information associated with the unique identity tag.

According to an aspect of some embodiments of the present invention there is provided a method of charging a customer for cryoprobe use, comprising:
a) supplying to a customer a plurality of cryoprobes, each associated with a unique identifying tag;
b) using a cryosurgery control module to record usage statistics for each of the cryoprobes when the cryoprobes are used; and
c) charging a customer according to the recorded usage statistics.

According to an aspect of some embodiments of the present invention there is provided a cryoprobe comprising an electronic module which comprises a memory and a communications interface.

According to an aspect of some embodiments of the present invention there is provided a cryotherapy system comprising
a) a cryoprobe which comprises
   i) a treatment head coolable by delivery thereto of a cryogen; and
   ii) an embedded electronic module which comprises a memory and a communication interface;
b) a cryogen supply; and
c) a cryogen control module operable to regulate flow of cryogen from the cryogen supply to the cryoprobe in response to information received from the embedded electronic module.

According to some embodiments of the invention the electronic module comprises a read-only memory which may comprise a unique identity code associated with the cryoprobe. The identity code may be reported by the communication interface to the control module when an electronic communications pathway is first established between the electronic module and the control module.

According to some embodiments of the invention at least one of a group consisting of the electronic module and the control module is programmed to record operational testing of the cryoprobe, and the control module is operable to prevent clinical use of the cryoprobe if the cryoprobe has not been operationally tested.

According to some embodiments of the invention at least one of a group consisting of the electronic module and the control module is programmed to record events of usage of the cryoprobe, and the control module is programmed to prevent supply of cryogen to the cryoprobe if more than a predetermined amount of usage has been recorded.

According to some embodiments of the invention the system further comprises, embodied in a common connector housing, a cryogen connector for connecting the cryogen supply to the cryoprobe and an electronic connector for connecting the control module to the embedded electronic module.

According to some embodiments of the invention a characterization of the cryoprobe is written into the memory of the electronic module during manufacture of the cryoprobe and is useable by the control module during algorithmic determination of operational parameters used during operation of the cryoprobe.

According to some embodiments of the invention operating values detected during testing of the cryoprobe are written into the memory of the electronic module and are useable by the control module during algorithmic determination of operational parameters used during operation of the cryoprobe.

According to some embodiments of the invention operating values detected during manufacture of the cryoprobe are written into the memory of the electronic module and are useable by the control module during algorithmic determination of operational parameters used during operation of the cryoprobe.

According to an aspect of some embodiments of the present invention there is provided a method for cryosurgery, comprising using a controller with a processor and a memory to algorithmically calculate commands controlling supply of cryogen to a cryoprobe insertable into a patient, the calculation being at least partially based on information read from a memory comprised in an electronic module embedded in the cryoprobe.

According to some embodiments of the invention, the information comprises at least one of a group consisting of:
a) a code uniquely identifying the cryoprobe;
b) information characterizing a usage history of the cryoprobe;
c) data derived from an operational test of the cryoprobe;
d) a type designation for the cryoprobe; and
e) a descriptive characterization of the cryoprobe.

According to an aspect of some embodiments of the present invention there is provided a method for regulating use of a cryoprobe, comprising:
a) recording a unique identification code in a read-only memory embedded in a cryoprobe;
b) recording an activity history of the cryoprobe by recording probe usage events associated with the unique identification code; and
c) regulating use of the probe by identifying the cryoprobe by reading the unique identification code from the read-only memory, and choosing between supplying cryogen to the probe and denying supply of cryogen to the probe, the choice being determined algorithmically as a function of a recorded probe activity history identified by the unique identification code.

According to an aspect of some embodiments of the present invention there is provided a method for cryosurgery, comprising:
a) recording, in an electronic module embedded in a cryoprobe, a history of usage of the cryoprobe; and
b) regulating use of the probe by choosing between supplying cryogen to the probe and denying supply of cryogen to the probe, the choice being determined algorithmically based on a reading of the recorded probe activity history.

According to an aspect of some embodiments of the present invention there is provided a method of doing business, comprising:
a) supplying to a customer a plurality of cryoprobes each of which comprises an electronic module which comprises a read-only memory holding a unique identity number;
b) using a cryosurgery control module to record usage statistics for each of the cryoprobes when the cryoprobes are used; and
c) charging a customer according to the recorded usage statistics.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse or a voice-control module are optionally provided as well.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a simplified schematic of an exemplary embodiment of a cryotherapy system according to an embodiment of the present invention;

FIG. 2 is a simplified schematic presenting details of an electronic module embedded in a cryoprobe of the system of FIG. 1, according to an embodiment of the present invention;

FIG. 3 is a simplified schematic of a connector comprising both gas conduits and electronic data lines, for connecting a cryoprobe both to a cryogen source and to a controller, according to an embodiment of the present invention;

FIG. 4 is an image showing a section of a cabinet for a cryogen supply and cryogen supply controller, comprising several sockets suitable for receiving the connector shown in FIG. 3, according to an embodiment of the present invention;

FIG. 5 is a simplified schematic of an extension cable utilizing comprising a plug as shown in FIG. 3 and a socket as shown in FIG. 4;

FIG. 6 is a simplified flow chart of a cryotherapy method, according to an embodiment of the present invention;

FIG. 7 is a simplified flow-chart of a method of doing business according to an embodiment of the present invention; and

FIG. 8 is a simplified schematic of a component of a cryosurgery system, according to an embodiment of the present invention;

FIG. 9 is a simplified flowchart of a method of use of a cryosurgery system, according to an embodiment of the present invention; and

FIG. 10 is a simplified schematic of a component of a cryosurgery system, according to an embodiment of the present invention.

### DESCRIPTION OF SPECIFIC EMBODIMENTS OF THE INVENTION

The present invention, in some embodiments thereof, relates to a cryosurgery system, and more particularly, but not exclusively, to a cryosurgery system incorporating a cryoprobe which comprises an electronic module.

In some embodiments, a cryoprobe according to the present invention comprises a treatment head operable to be cooled to cryoablation temperatures, and further comprises an electronic module which comprises a memory. In additional embodiments a cryoprobe according to the present invention comprises a treatment head operable to be cooled to cryoablation temperatures, and further comprises a response module operable to receive a query signal from a controller and to send a response signal in response to said query signal. Some embodiments comprise both a memory and a response module as defined in detail herein below.

As used herein, the term “cryosurgical probe” is used to refer to a probe which is either a cryoprobe operable to cool tissues of a body, or another type of probe (without cooling capabilities) which is insertable in a body and useable in conjunction with a cryoprobe during a cryosurgical procedure. In some embodiments, a cryoablation system according to the present invention comprises one or more cryoprobes which comprise an electronic module having a memory, a cryogen supply, and a controller operable interact with electronic module(s) of the cryoprobe(s) and further operable to control delivery of cryogen from the cryogen supply to the cryoprobe(s). In some embodiments the controller is programmed to read data from the electronic module memory (or memories) and to calculate and execute commands controlling flow of cryogen to the cryoprobe, the calculations being at least partially based on that read data. Such systems may optionally comprise additional types of cryosurgical probes, some of which may also comprise electronic modules operable to interact with the system controller.

In some embodiments the electronic modules of the cryosurgical probes are embodied as chips embedded in the probes. In an exemplary embodiment presented in detail below, an electronic module is embedded in a proximal portion of a cryoprobe near or in a connector by which the probe is connectable both to a cryogen source and to a system controller operable interact with (e.g. read data from and optionally write data to) the electronic module in the probe. In this exemplary system, the controller is operable to calculate commands for controlling flow of cryogen from cryogen supply to cryoprobe, and optionally also for controlling supply of heat sources, the calculations being at least partially based on data read from a memory comprised within the electronic module embedded in the cryoprobe.

Optionally, the cryoprobe is manufactured with a unique identifying code written into a read-only memory of the electronic module. Read-only and/or read-write memories incorporated in the electronic module may be used to store, within the probe, that unique identifying code and/or a variety of other probe-descriptive data. This data can be read (and optionally updated) by the system controller. The controller of this exemplary embodiment can use data read and optionally written to the probe to manage probe usage, enforce safety standards, enhance reliability of the cryoablation system, and/or to enable simplified automated control of a plurality of probes used simultaneously, including for example verification that characteristics of probes connected to the controller correspond to types and characteristics called for in a surgical plan, and/or adjustment of cryogen supply to each probe as a function of known characteristics of that probe. Using a cryoablation system as herein described, theoretical probe specs and/or measured probe characteristics, written to the probe memory, can conveniently be read therefrom and be taken into account in planning and executing surgical operations. Using such information, mixtures of probes having differing operating characteristics can conveniently be used together and be appropriately individually controlled by a common controller. Using such information, usage limitations based on safety standards or commercial considerations can be enforced. The system further enables to manage commercial arrangements (e.g. methods for billing based on actual probe use) which would not otherwise be practical.

In additional embodiments, a system comprises one or more cryoprobes with coolable treatment heads, which cryoprobes also comprise a response module operable to receive a query signal from a controller and to send a response signal in response to said query signal. The system also comprises a cryogen control module (also referred to as a “controller” herein and in the claims below) which is operable to send an inquiry signal to the cryoprobe(s), receive a response signal send from the cryoprobe in response to the inquiry signal, and, by analyzing that response signal with reference to the query signal it answers, uniquely identify the cryoprobe sending the response. The controller optionally comprises a memory for recording information about the uniquely identified cryoprobe(s), a cryogen supply, a cryogen flow control mechanism for regulating flow of cryogen from a cryogen supply to the cryoprobe(s), and a calculation module for calculating cryogen flow commands which influence operation of the cryogen flow control mechanism, said calculation being based at least in part on information associated with said uniquely identified cryoprobe and stored in the memory.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

It is expected that during the life of a patent maturing from this application many relevant cryoprobes and cryosurgery probes will be developed and the scope of the terms “cryoprobe” and “cryosurgery probe” are intended to include all such new technologies a priori. Additionally, it is expected that during the life of a patent maturing from this application many relevant techniques for incorporating an electronic module in a probe, and many forms chips, of electronic modules and of electronic memories will be developed. The scope of the terms “electronic module” and “memory” are intended to include all such new technologies a priori.

The terms “comprises”, “comprising”, “includes”, “including”, “having” and their conjugates mean “including but not limited to”.

The term “consisting of means “including and limited to”.

As used herein, the singular form “a”, “an” and “the” include plural references unless the context clearly dictates otherwise.

In discussion of the various Figures described herein below, like numbers refer to like parts.

The drawings are generally not to scale.

For clarity, non-essential elements were omitted from some of the drawings.

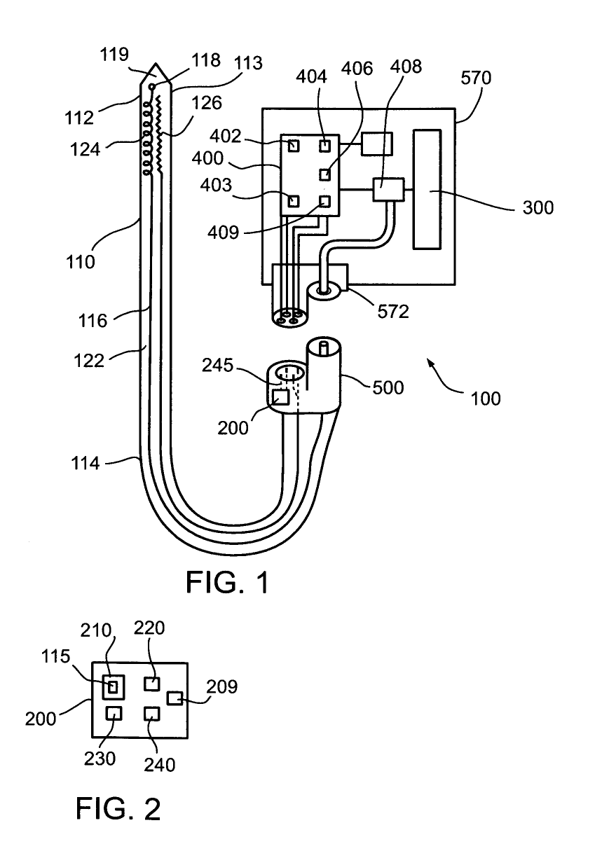

Referring now to the drawings, attention is drawn to FIG. 1 which presents a simplified schematic of an exemplary embodiment of a cryotherapy system according to the present invention. FIG. 1 presents a cryotherapy system 100 comprising a cryoprobe 110 which comprises a treatment head 112 coolable by delivery thereto of a cryogen, a cryogen supply 300 for supplying a cryogen to probe 110, and a controller 400 for controlling delivery of cryogen from supply 300 to cryoprobe 110. Cryoprobe 110 comprises an electronic module 200. In this non-limiting exemplary embodiment supply 300 and controller 400 are housed in a common cabinet 570. A connector 500 is provided on a proximal portion of probe 110 for connecting probe 110 to a socket 572 on cabinet 570, providing a gas connection to supply 300 and electrical/electronic connection to controller 400.

In the exemplary embodiment shown in FIG. 1, cryoprobe 110 has a distal portion 113, a flexible hose portion 114 and a proximal connector 500. A cryogen supply conduit 116 supplies a cryogen (high-pressure cooling gas such as argon, or another cryogen) to a Joule-Thomson orifice 118 in an expansion chamber 119 in a treatment head 112. A cryogen exhaust conduit 122 carries expanded gas away from head 112. A heat exchanger 124 positioned in or near head 112 provides for pre-cooling of high-pressure gas approaching head 112. It is however to be understood that although Joule-Thomson cooling is presented in this exemplary embodiment, cooling by evaporation of a liquefied cryogen, or any other form of cooling, may also be used within the scope of the present invention.

Optionally, in this exemplary embodiment, an electrical heater 126 may be integrated with heat-exchanger 124, or may be positioned elsewhere in probe 110, to -.provide optional heating of head 112. Alternatively, a heating gas such as high-pressure helium may be supplied by controller 400 and supply 300, to heat head 112 to facilitate disengagement after freezing, or for other purposes. Further alternatively, no heating may be provided.

As mentioned above, probe 110 comprises electronic module 200. In this exemplary embodiment module 200 is shown as embedded within connector 500, yet is should be understood that module 200 may be positioned anywhere in or on any part of probe 110, according to convenience of manufacture and/or convenience of use.

Attention is now drawn to FIG. 2, which presents additional details of electronic module 200, according to an embodiment of the present invention. Module 200 comprises a read/write memory 210 and/or a read-only memory 220, a communication interface 230 and may comprise a processor 240 and/or additional electronic components 209 (e.g. a sensor and/or a timer and/or an analog/digital converter). Communication interface 230 provides a data transfer path between memories 210/220 and controller 400. Power and data links 245 (shown in FIG. 1), which may be a combined power and data link, enable connecting module 200 to controller 400 through connector 500 and socket 572. An exemplary embodiment of module 200 comprises an EEPROM such as model DS2433 4kb 1-Wire EEPROM from Dallas Semiconductor.

Referring again to FIG. 1, cryogen supply 300 supplies a cryogen to probe 110. This cryogen may be a gas such as high-pressure argon or another high-pressure cooling gas, or may be a liquefied gas operable to cool by evaporation, or may be any other cryogen. Cryogen supply is controlled by controller 400. Optionally, supply 300 is also optionally able also to supply high-pressure helium or another heating gas, to be used for heating portions of probe 110. Further optionally, supply 300 may be equipped to supply an electrical current useable by an electrical heating element 126 useable to heat portions of probe 110.

Controller 400 may comprise a memory 402, a processor 404, and a user interface 406. In this exemplary embodiment controller 400 controls flow of cryogen from cryogen supply 300 to cryoprobe 110 using servo-controlled valves 408, in a manner well known in the art. In some embodiments controller 400 is programmed to regulate the flow of cryogen from cryogen supply 300 to cryoprobe 110 in response to information received from memories 210 and/or 220 of module 200. Optionally, controller 400 may be further programmed to regulate the flow of a heating gas from supply 300 to probe 110, and/or to regulate a flow of electric current to electric heater 126 within probe 110. Controller 400 may be programmed to calculate and issue commands in response to information received from memories 210 and/or 220 of module 200 and/or in response to information received from one or more sensors within probe 110 or otherwise connected to controller 400 or communicating with controller 400, and/or in response to commands issued by an operator and/or in response to communications from a remote source received by a remote-communications module 403 within controller 400.

Memory 402 may be physically joined with or contiguous to other portions of controller 400, or optionally may be physically distant therefrom, for example a memory accessed through a network (such as a hospital network) or through the internet.

Controller 400 is operable to read information from memories 210 and/or 220 of module 200 and optionally is operable to write information to memory 220 of module 200. Read-only memory 220 contains information written into it during manufacture and/or factory calibration but not modifiable during use. In some embodiments, during manufacture each memory 210 is made to contain a readable unique identity code associated with the particular probe 110 into which that code is placed. Consequently, data in memory 210 of module 200 may be used by controller 400 to identify individual cryoprobes by their unique identity codes. In addition, probe descriptions and characterizations (e.g. probe types) and empirical probe characterizations (e.g. probe specs or probe usage test results) may also be written into memories 210 and/or 220. Such information, readable by controller 400, enables controller 400 to use identifying information and/or probe characterization information to control probe use, and to enabling probe use planning and/or real-time probe use functional calculations, based on empirically measured probe characteristics read from the probe memory. Controller 400 can also record and report individual and collective probe usage statistics, can manage billing of clients according to actual probe use, can limit or otherwise regulate probe re-use for commercial purposes and/or to enforce safety standards or for other clinical purposes, and in general can monitor, report, and control probe use. Testing status, measured operating statistics, activation history, and other information written into memories of module 200 and read by controller 400 can be algorithmically treated by controller 400 to enable/disable use of individual probes 200 and/or to provide for clinical use of probe 200 according to individually tailored operating parameters based on recorded test results or other recorded probe-specific information.

The capabilities mentioned in the preceding paragraph and elsewhere herein constitute a potential advantage of probe 110 and system 100 over prior art probes and cryosurgery systems. For example, some cryoprobe manufacturers instruct users to test probes prior to use, and to avoid excessive re-use, and users may even undertake an obligation to quantitatively limit probe re-use, yet prior art systems provided no means for verifying such user behavior nor for enforcing these limitations. As shown above, means for such verification and enforcement may be provided by system 100. System 100 is optionally operable to ensure that only probes manufactured to be compatible with controller 400 will be useable with controller 400. Optionally, controller 400 may further comprise a remote-communications module 403 for communicating with a remote server, such as a server accessible through the Internet or by other communication means and run by a manufacturer of system 100 or by a commercial intermediary such as a local supplier of system 100. Such communications may be used to report probe usage patterns, to request and receive authorization for an operation, for inventory management, for automated billing, or for other purposes.

It is noted that additional electronic components 209 and 409 may optionally be installed in module 200 and controller 400 respectively, to provide additional functionality. For example, component 209 and/or 409 might comprise an analog to digital converter. Such a component could be used, for example, as part of a temperature-reporting system wherein a current meter or voltage meter or resistance meter is provided to assess the temperature of a resistive heater as a function of the heater's electrical characteristics. Other forms of temperature sensors can also be digitally interfaced, through module 200, to controller 400. A pressure sensor, flow meter, or other sensor may similarly be included and so interfaced. In another example, components 209 and 409 might comprise radio frequency communications devices or other communications devices enabling wireless communication between module 200 and controller 400.

Cabinet 570 may enable simultaneous connection and controller 400 may enable simultaneous control and use of a plurality of probes 110. (For simplicity of the Figure, only one such connection is shown in FIG. 1.) In some embodiments controller 400 can verify the identity and type of connected probes by reading probe memories as explained above, and can modify operating parameters (e.g. time and pressure of cryogen supply) of each connected probe, taking into account probe-specific recorded information. These capabilities enable controller 400 to tailor such parameters as cryogen pressures and flow times to individual probes or groups of probes, thereby facilitating simultaneous use of a pluralities of differing types of probes with a same controller during a same operation. Verification that probes actually connected correspond to those whose connection was planned or intended is an additional safety feature provided by system 100.

As mentioned above, electronic module 200 may be used to identify cryoprobe 110. In some embodiments identification of probe 110 is based on a unique identification code 115 written into read-only memory 210 during manufacture, and which may be read out of read-only memory 210 during power-up (e.g. at the time of initial connection electronic connection between probe 110 and controller 400), or at any other time. Read-out of this probe-specific identifying code can be used to maintain a record of probe usage history outside of probe 110, e.g. in a memory 402 of controller 400. Using techniques well known in the art, code 115 may be generated having identifiable characteristics which can be used by controller 400 to determine that a given probe, connected to system 100, is compatible with operating requirements of system 100. These requirements may include, in addition to physical characteristics of the probe 100, such characteristics as an identification as being supplied by a particular manufacturer. Thus, probe-specific characterization, probe sources or other commercial status information, probe-specific manufacturing and test information, probe operating histories and similar information may be recorded in controller 400, based on information read from individual probes 110. Alternatively or additionally, such probe-specific information can be recorded within the probe in one or both memories of module 200.

Additionally, general statistical information relevant to a plurality of probes connected (sequentially or simultaneously) to controller 400 may be maintained in or reported by controller 400.

In one form of use, controller 400 may be programmed to prevent clinical operation of a specific probe 110 unless or until that probe 110 is known (e.g. according to a history recorded within the probe, or according to a history recorded in controller 400 in a record associated with that probe 200's unique identification number) to have successfully passed a pre-clinical testing protocol.

Similarly, probe specs and/or actual test measurements of operating characteristics of each probe 200 may be recorded within the probe or in a memory of controller 400 in a record associated with the probe's identification number, and such operating characteristics may subsequently be used by controller 400 to algorithmically calculate operating parameters to be used in operating the specific probe in view of a specific treatment plan. For example, the actual gas throughput of individual probes under identical cryogen pressure conditions will vary somewhat. Resultant operating characteristics (e.g. cooling capacity) of individual probes may be testing by testing operation under standard conditions and recording temperature results measured by sensors inside and/or outside the probe under standard conditions. This information may be recorded in module 200 of each individual probe or may be maintained in a memory of controller 400 as discussed above, and that information may then be used by algorithms of controller 400 to determine optimal operating parameters (e.g. length of timed cooling operations) of the probe according to a cryotherapy planning module.

Collection and use of such information will provide a more accurately determined cooling effect than will operation of probes merely according to the theoretical cooling capacities or other characteristics determined only by their intended manufacturing parameters.

An additional optional use of system 100 is to record operational testing parameters of individual probes and to program controller 400 to prevent accidental and/or intentional clinical use of cryoprobes which have not been operationally tested.

An addition optional use of the system described above is to record events of usage of cryoprobe 200, and to have control module 400 prevent supply of cryogen to any cryoprobe 200 if more than a predetermined amount of usage has been recorded, thereby providing a safety check to prevent excessive and unsafe repeated use of an individual probe by limiting the amount of repeated use to a predetermined amount.

An additional optional use of the system described above is to record events of usage of cryoprobe 200, and to have control module 400 report such use as a basis for charging a customer. In this method of business, cryoprobes can be supplied to customers without charge or with a fixed minimal charge, and additional charges can be levied according to recorded cryoprobe usage. It is a potential advantage of this system that customers can be supplied with a sufficiency of probes and a variety of probes of varying types and sizes, and the supplier can be compensated according to actual probe usage. In this context it is to be noted that read-only memory 220 may present probe type information as well as unique probe identity code, thereby enabling recording of statistical and business information pertaining to amounts of use of varying types of probes.

An additional optional use of the system described above is to facilitate use of a mixture of cryoprobes of differing capacities simultaneously or sequentially with a common controller 400. Since each probe supplies self-descriptive information to controller 400, controller 400 can be programmed to adapt its operational parameters to each probe individually, thus enabling to mix a plurality of probes with differing cooling capacities or other differing operational characteristics and yet easily cause each probe to conform to a pre-determined common cooling plan (e.g. a planned ice-ball shape and size) under algorithmic control. The system may optionally also be used to determine whether characteristics of probes actually connected for use correspond to probe characteristics called for in a surgical plan, thereby assuring that correctly characterized probes are inserted and used.

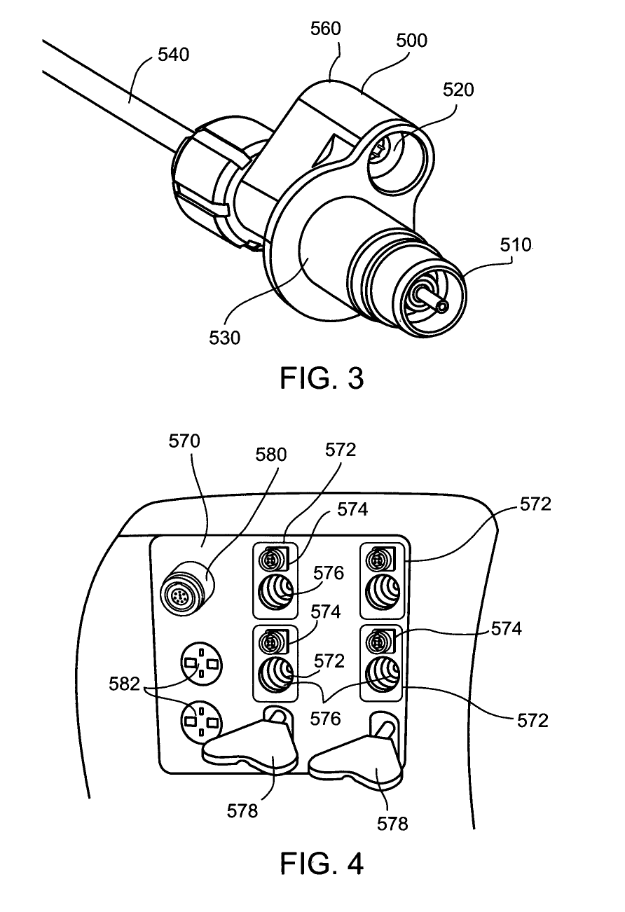

Attention is now drawn to FIG. 3, which shows an additional view of an embodiment of a connector 500 for connecting probe 110 to cryogen source 300 and to controller 400, according to an embodiment of the present invention. In a convenient embodiment shown in FIG. 3, a cryogen connector 510 and an electrical/electronic connector 520 may be combined in a common housing 530 to form a combined connector 500 by which a cryoprobe 200 may be connected to a combined socket 572 in a cryogen supply cabinet 570, which cabinet contains both cryogen supply 300 and controller 400, so that one act of “plugging in” probe 110 establishes both the cryogen supply connection between probe 200 and supply 300, and electronic data connection between probe 200 and controller 400.

Cryogen connector 510 may comprise, as shown in FIG. 3, a co-axial connector which comprises a central high-pressure conduit surrounded by a low-pressure gas return conduit.

Electronic connector 520 may comprise a plurality of pins insertable into corresponding sockets, for establishing data connection between module 200 and controller 400, optionally for establishing further data connections between controller 400 and sensors within probe 110, and optionally for establishing electrical power connections (e.g. for supplying power to a heater 126), and for any other purpose. It is noted that probes which are not themselves cryoprobes may also be connected through connectors 520 without cryogen connectors 510, so as to provide e.g. a data connection path for thermal sensor probes comprising one or more thermal sensors, and a data connection and/or electricity supply connection for a heating probe.

Sensors (e.g. temperature sensors, flow meters, pressure sensors) and/or an electrical heating element 126 incorporated in probe 110 may be connected to controller 400 through electronic module 200, or may be connected or directly to controller 400 through connector 500.

Shaft 540, shown in FIG. 3, extends the cryogen connection 510 and optionally the data connection 520 to a distal portion of probe 110, which distal portion is not shown in FIG. 3.

Optionally, a special embodiment of connector 500 labeled 560 may be provided. Connector 560 is a “service key” connector, which simulates a connector 500 in that it is compatible with a socket 572 (shown in FIG. 4), yet optionally does not comprise shaft 540 nor more distal portions of a cryoprobe. Connector 560 does comprise an electronic module 200 encoded in a manner which identifies connector 560 as a service key. In an exemplary embodiment service key 560 can be used to override various limitation or restrictions programmed into controller 400, for purposes of testing of controller 400, testing of sockets 572, calibration, and for other maintenance or commercial uses. Controller 400 is optionally programmed to recognize a service key 560 and to modify its responses appropriately when presence of an inserted service key 560 is detected.

Optionally, service key 560 may comprise information which, when read by controller 400, modifies the programming of controller 400 or modifies data held by controller 400 which influences controller 400 behavior while service key 560 is connected and/or after service key 560 is disconnected. Service key 560 may serve as a means of updating controller 400 and as a means for influencing controller 400 behavior after service key 560 is removed. Among other optional uses of service key 560, key 560 can be used to change limitations imposed by controller 400 on cryoprobe use. In particular, a key 560 can be used to cause controller 400 to enable use of a cryoprobe which is lacking an electronic module 200 or which comprises an electronic module not recognized by the system. An optional commercial use of this system is to enable to sell to a client a permission to use an unrecognized probe (e.g. a probe sold by another supplier) with controller 400, by supplying to the user a service key 560 which communicates this permission to controller 400.

Attention is now drawn to FIG. 4, which shows a section of a cabinet 570 comprising several sockets 572, suitable for receiving the embodiment of connector 500 shown in FIG. 3, according to an embodiment of the present invention.

As shown in FIG. 4, socket 572 may comprise a socket 574 for receiving electrical power and electronic data connections from connector 520. The male portion (pins) of the data connection are typically more fragile than the corresponding pin sockets. In this exemplary embodiment pins are provided on the probe 110 side of the connection (probes being optionally disposable) rather than on the multiply-reusable cabinet 572 side of the connection.

Socket 572 may also comprise a socket 576 for receiving coaxial cryogen connector 510. In an exemplary embodiment of system 100, each socket 576 comprises a high-pressure gas line which is individually controlled by two gas valves (not shown), one of which controls delivery of a high pressure cooling gas such as argon, and a second which controls delivery of a high pressure heating gas such as helium or of a low pressure gas (which can be a low pressure cooling gas) which may be heated in cabinet 570 and/or by heater 126. Controller 400 and connector 572 are optionally designed to prevent escape of gas from gas supply 300 by closing the supply valves 408 if no probe 110 is connected to a connector 572.

Additional optional features of the exemplary embodiment shown in FIG. 4 include
A group lock, here embodied as a turnable “butterfly” key 578, useable to lock a plurality of connectors 500 to cabinet 572. In the embodiment shown in FIG. 4, each key 578 locks a group of two connectors 500. In another exemplary embodiment each key locks five connectors 500, and other group sizes and combinations may be used. In some embodiments, each such group of connectors is serviced by a common pair of heating and cooling gas valves 408. Alternatively, each connector, and consequently each probe 110, may be individually controlled.
A multi-pin connector 580 useable for connecting a multi-sensor thermal sensor probe, which probe may also comprise an electronic module 200.
Connectors 582 for connecting individual thermocouple sensors, which may also comprise an electronic module 200.

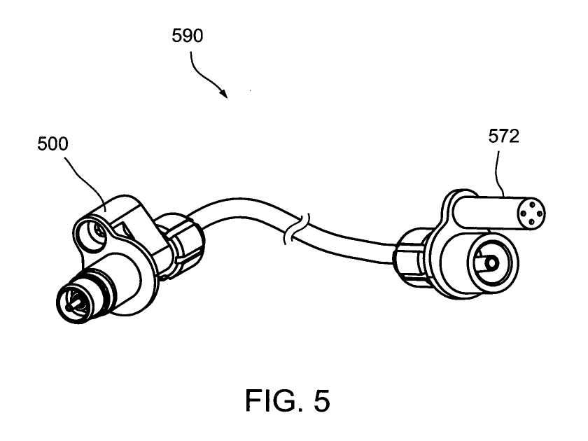

Attention is now drawn to FIG. 5, which shows an extension connector 590 which can be used to provide an extended-length connection between a probe 110 and cabinet 570, according to an embodiment of the present invention. Extension 590 comprises a connector 500 on one end and a compatible socket 572 on another end. Extension 590 is useful when a particularly long distance separates cabinet 570 from a point of use of probes 110, as may be the case, for example, when probes 110 are to be used within an MRI magnetic environment and controller 400 and cryogen supply 300 are maintained outside that magnetic environment.

It is to be noted that characteristics of the particular exemplary embodiment presented in the Figures are not to be understood as limiting. For example, FIGS. 3 and 4 and the text describing them refer to common connectors 500 and 572 useful for connecting a probe 110 to a cabinet 570 which houses controller 400 and cryogen supply 300, yet it is noted that controller 400 and cryogen supply 300 may be housed separately and connections thereto may be made separately and not by means of a common connector 500 combining connectors 510 and 520. It is further noted that if components 209 and 409 comprise wireless connections, then data links 245 and wire connections 520 and 574 may be absent.

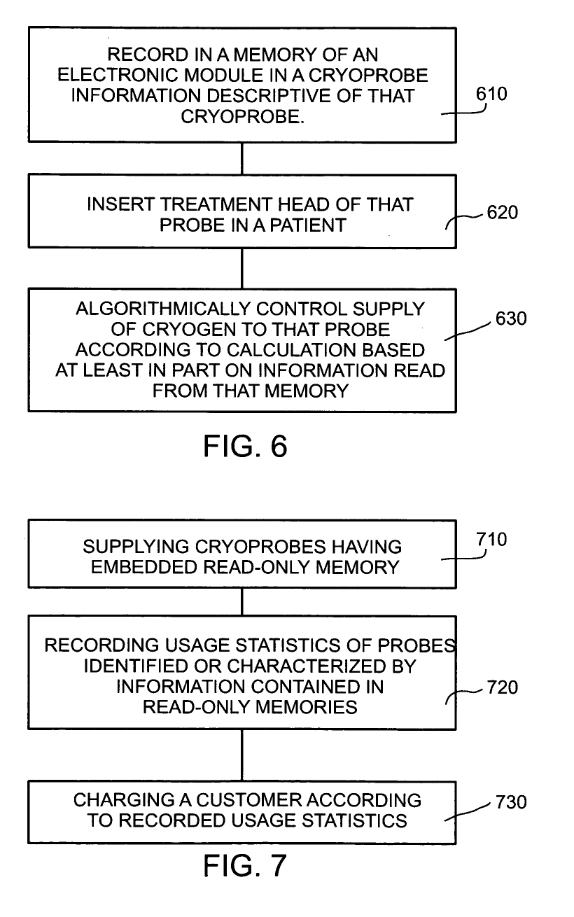

Attention is now drawn to FIG. 6, which is a simplified flow chart of a cryotherapy method, according to an embodiment of the present invention. A cryotherapy method as shown in FIG. 6 comprises, at 610, recording in a memory comprised in an electronic module embedded in a cryoprobe information descriptive of that cryoprobe, at 620, inserting a distal treatment head of that cryoprobe in a patient, and at 630 algorithmically calculating commands regulating supply of cryogen to that cryoprobe, the calculation being based at least in part on information read from that electronic module memory.

Information recorded in the probe at 610 may include (but is not limited to), one or more of:
A unique identification code;
A type identification code identifying probe type or other characterizing information; and
An activity history of the probe, such as a listing of probe testing events and test measurements and/or probe usage events.
   It is noted that such recorded information may be recorded in a memory 210/220 of the probe, and that alternatively such information may be recorded in a memory external to the probe (e.g. in memory 402 of controller 400), in a record associated with the probe's unique identification code (or other probe-identifying information), which probe-identifying information is recorded in probe memory 210 and/or 220.

Regulation of probe usage at 630 may include
Supply or denial of supply of cryogen as a function of recorded probe characteristics; and
Modulation of cryogen supply or of heating as a function of recorded probe characteristics.

Information may be recorded in read-only memory 220 (e.g. memory writable by the manufacturer but only readable and not writable by an end user), and/or in read-write memory 210. Information may be recorded during manufacture, during factory testing and factory calibration, during packaging (e.g. packaging into kits containing a set of probes intended for a single surgical operation), during pre-operation testing at a hospital, during a surgical procedure, and post-operatively. Information recorded in memories 210 and/or 220 may be transmitted to controller 400 when probe plug 500 is successfully plugged into a socket 572, or may be transmitted to controller 400 upon receipt of an electronic query from controller 400, or on detection of a triggering event by a timer or sensor included in module 200.

Attention is now drawn to FIG. 7, which is a simplified flow-chart of a commercial activity according to an embodiment of the present invention. The method comprises, at 710, supplying to a customer a plurality of cryoprobes each of which comprises probe-identifying and/or probe-characterizing information readable from a read-only memory comprised in an electronic module embedded in each probe; at 720 using a cryosurgery control module which comprises a processor to record usage statistics for each probe attached to the control module and supplied with cryogen under control of said control module; and at 730 charging a customer according to said recorded usage statistics.

Additional features of some exemplary embodiments of the invention include the following:
Pre-defined default operation (e.g. stoppage of cryogen flow) on detection of communication failure or other electronic/computational errors.
CRC or similar checking of communications between module 200 and controller 400 to ensure accurate communications and protocols to prevent or stop potentially dangerous cryoprobe operations in the event of communications failures.
Preventing use of cryoprobes having exceeded a predetermined criterion of amount of use, which may be implemented as follows: a predetermined maximum count of freeze cycle activations may be written into cryoprobe memory and decremented once per activation, or once per change of activation state (e.g. from one temperature range or cryogen pressure range to another), or once per pre-determined period of time (e.g. 5 minutes), or according to some other criterion. In an exemplary method of use, controller 400 decrements the count according to one or more of these criteria, provides warnings to an operator as the decremented count nears zero, and disallows further activation when the decremented count reaches zero. Optionally, continuation of probe use might be allowed in a manner which does not interrupt an ongoing clinical procedure, yet which would not allow a probe with a fully-decremented count to be used during an additional clinical procedure.

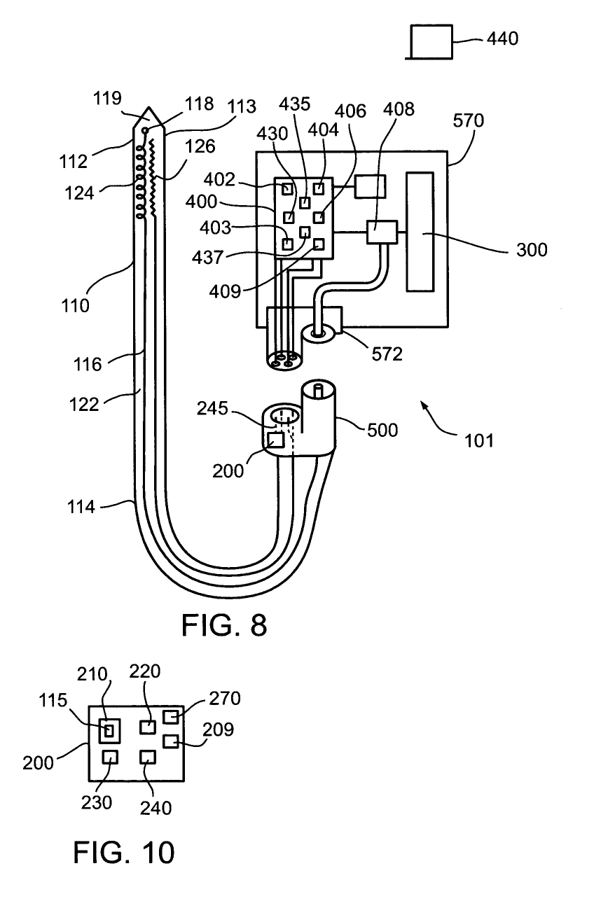

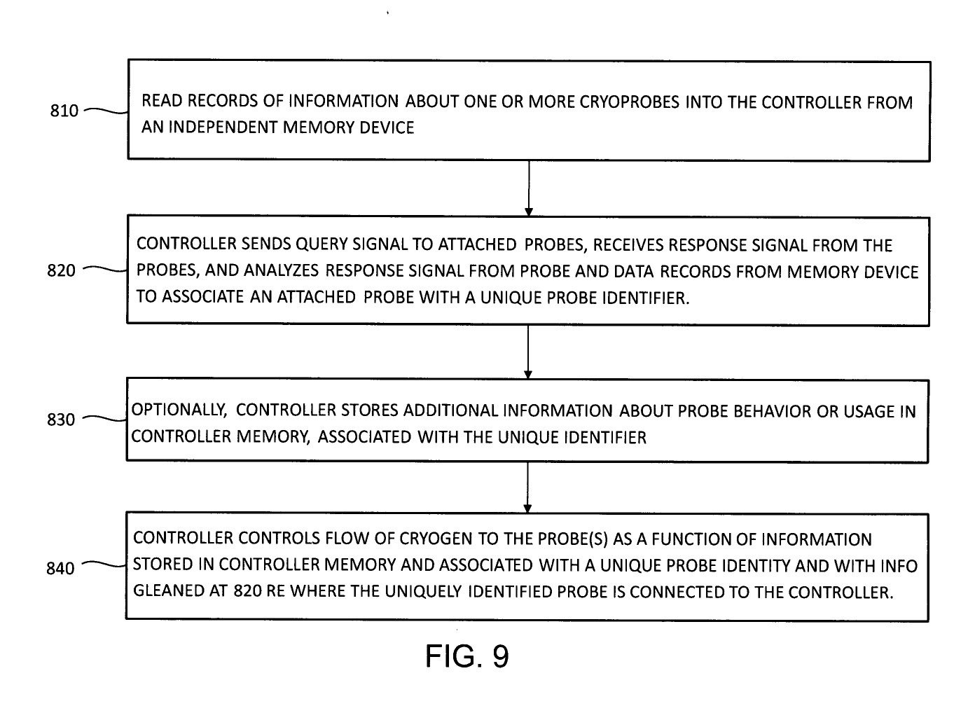

Attention is now drawn to FIGS. 8-10, which present additional exemplary embodiments and/or features labeled system 101. FIGS. 8 and 10 are simplified schematics of components of cryosurgery system 101, according to an embodiment of the present invention, FIG. 10 presenting a detail of cryoprobe 110 of FIG. 8. FIG. 9 is a simplified flowchart of a method of use of system 101, according to an embodiment of the present invention.

System 101 can be similar to system 100, and most of the functionality and methods taught above with respect to system 100 can be present and/or available in system 101. The two systems are distinct in that system 101 does not necessarily comprise memory modules 210 and 220. System 101 uses an alternative system for uniquely identifying cryoprobes of the system, and in some embodiments most or all of the functions requiring recording of information in a memory take place in controller 400 and not in module 200 within the cryoprobe.

System 101 comprises the following components:
Controller 400 comprises a data reader 430, and at least one data memory source 440. Data memory source 440 may be a simple memory device such as a plug-in “memory stick” with a read-only memory or a read-write memory, or it may be any other device useable to transmit information to system 101 detailing something known about at least one cryoprobe 110. For example, data source 440 might be a portable device such as a card with a magnetic strip or a barcode, or a diskette or a CD, on which information on cryprobes 110 has been recorded. Alternatively, device 440 might be an interface for receiving such information over a network or over the internet.
Controller 400 further comprises a query module 435. Query module 435 functions to formulate, based on information received from data source 430, a query for sending to cryoprobe 110.
Controller 400 optionally further comprises an identifier module 437, for receiving from cryoprobe 110 a response to a received query signal, and for analyzing that response signal to determine if it is possible, based on that signal, to establish a unique identity tag for cryoprobe 110. If so, then that identity tag is made known to other portions of controller 400, in particular to enable data records read by module 430 from source 440 and data records read from any other sources during use or testing of probe 110 to be identified with a specific, uniquely identified cryoprobe 110.

Attention is drawn to FIG. 9, which is a simplified flowchart of a procedure for establishing a unique identity tag for probe 110, according to an embodiment of the invention. At 810, records of information about one or more cryoprobes are read into controller 400 from an independent memory device 440. Device 440 might, for example, be a data card or data disk supplied to a user as part of kit which comprises a number of probes 110 intended for a surgical procedure. The information on device 440 might include any of the information discussed hereinabove as characterizing cryoprobes, such as manufacturing specifications, empirical test data generated during manufacture, limitations of use of the probe, previous usage history of the probe, or anything else.

At this point, controller 400 holds information about one probe, or more typically a plurality of probes, such as for example probes supplied together in a kit of probes, or perhaps probes from several kits. It is recalled that in system 100 a memory module from probe 110 supplies to controller 400 a unique ID code, and that code is associated with the probe which supplied it. In contrast, in system 101 (at 820 of FIG. 9) the method is different: based on information about the probes obtained from source 440 and read by controller 400 through input device 430, controller 400 uses query device 435 to calculate a query signal based in information from device 440, and sends it to cryoprobe 110. In a simple embodiment, the query signal might simply be an identity code for a probe read from device 440. Alternatively the query signal might be the result of an algorithmic calculation based on an identity code or on other information.

Query module 435 may calculate and send a query when probe 110 is first connected, or at any other time. Optionally, query module 435 sends a series of query signals to probe 100, the series based on information known to controller 400 about one or more cryoprobes.

Cryoprobe 110 comprises a response module 270 which receives the query and responds. In the simple embodiment mentioned above, wherein query signals are unique probe identifiers, response module 270 simply tests an incoming signal to determine whether the incoming signal is recognized as its own unique identifier. If so, module 270 sends a “yes” response, which can be an encoded signal or a simple signal. If not, module 270 sends a “no” response or no response. In the event of a “no”, query module 435 then sends other queries based on information about other cryoprobes in its data list (input from source 440 or any other source), cycling through its list of known cryoprobes until a match is found. If module 270 sends a “yes”, then the probe is identified and the query process terminates.

According to system 101, probe 110 does not send to controller 400 any information specifically read from a memory in probe 110, indeed in some embodiments probe 110 may not have a memory as such. Probe 110 does, however, optionally send information generated in probe 110 in response to a query, and that response enables controller 400 to determine whether probe 110 is or is not the uniquely identified probe on whose stored information the query is based.

Alternatively, response module 270 might comprises a small processor operable to perform an algorithmic calculation. For example, module 270 might be what is called a “random number generator” able to generate a pseudo-random number based on a seed, or a module operable to perform any other mathematical function based on a received operand. In this scenario, query module 435 sends an operand, response module 270 calculates a response as the value of its embedded function and sends it back, and identification module 437 analyzes the response to determine if the response was as expected, in terms of the data known to controller 400. (In some embodiments, identification module 437 simply performs that same calculation as is done in response module 270, and declares a “yes” if the result of the calculation in the controller is identical to the result of the calculation in the response module.) If so, this constitutes a “yes” response. If not, this constitutes a “no” response. In this manner query module 435 can run through information based on all the cryoprobes known to it from data input 440, checking probe responses until a “yes” response is received, or until a function response calculated by identification module 470 to be a “yes” response is received.

In general, in some embodiments information read from device 440 comprises a code which, when sent to said cryoprobe in an inquiry signal, will provoke a response signal which uniquely identifies the cryoprobe.

At that point, controller 400 knows which of the cryoprobes known to it is attached at the position to which the queries are sent. From then on, the various procedures and methods outlined above with respect to system 100 can be undertaken, as shown at 830 and 840 of FIG. 9. Information read from device 440 and now associated with a particular connected cryoprobe can include information characterizing a usage history of said cryoprobe, data derived from an operational test of the cryoprobe, a type designation for the cryoprobe, and a descriptive characterization of the cryoprobe.

In particular, probe test results and probe usage data can be recorded in a memory of controller 400 as associated with the unique identity code of a cryoprobe recognized and identified according to the methods shown in FIG. 9 and described above.

In particular, it is noted that in system 101, controller 400 can record information attesting to a cryoprobe 110 having undergone operational testing, and can prevent clinical use of the cryoprobe if such information has not been so recorded.

In system 101, controller 400 can be programmed to record events of usage of cryoprobes 110, and to prevent supply of cryogen to a cryoprobe if more than a predetermined amount of usage has been recorded. In system 101, controller 400 can receive and record sensor values detected during testing of a uniquely identified cryoprobe, and can calculate cryogen supply parameters for use during operation of the cryoprobe as a function of the recorded values.

In an exemplary embodiment of the invention, system 101 enables use of a method for regulating use of a cryoprobe, comprising:
a) reading information descriptive of a cryoprobe from a portable memory device into a controller;
b) establishing a unique identity tag with said cryoprobe by
   i) sending an inquiry signal based on that read information to a cryoprobe,
   ii) receiving a response signal from the cryoprobe in response to said inquiry signal; and
   iii) associating said read information with said cryoprobe and with a unique identity tag for that cryoprobe if and only if the response signal conforms to predetermined criteria.
Optionally, the method also comprises recording an activity history of the cryoprobe by recording probe usage events related to use of the cryoprobe in a memory record associated with a unique identity tag; and regulating use of the cryoprobe by calculating cryogen flow commands as a function of recorded information associated with the unique identity tag.
   In an exemplary embodiment of the invention, system 101 also enables use of a method of doing business, comprising:
a) supplying to a customer a plurality of cryoprobes, each associated with a unique identifying tag;
b) using a cryosurgery control module to record usage statistics for each of said cryoprobes when said cryoprobes are used; and
c) charging a customer according to said recorded usage statistics.

In some embodiments all or most data recording and probe management is undertaken by controller 400, but it should be understood that this is not necessarily a limitation of system 101. Probes 110 of system 101 may contain electronic modules with probe memories and computational ability beyond that described herein for response module 270. Some embodiments of system 101 do. Some embodiments of system 101 do not.

Some variants of system 101 are now considered.

Response module 270 may comprise a calculator operable to calculate its response signal as a mathematical function of a value presented by the inquiry signal. Alternatively, it may calculate its response signal as a mathematical function without an operand, in response to a query signal not used as an operand to the function. Optionally, response module 270 can be an analog electronic circuit. Optionally, module 270 can be an embedded radio-frequency (RF) tag.

Response module 270 may be operable to recognize when a received query signal possesses a predetermined characteristic, and to emit a characteristic response when an inquiry code having said predetermined characteristic is recognized. A query signal presenting a unique cryoprobe ID code and a response module which responds “yes” if it recognizes that code is an example. For another example, a response module might have a memory containing an expiration date, a query signal might be recognized as supplying a real-time date and asking for a response from probes whose expiration date is prior to that real-time date, and response module 270 might present a “yes” or “no” response accordingly.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A cryotherapy system comprising
a) at least one cryoprobe which comprises
i) a treatment head coolable by delivery thereto of a cryogen; and
ii) a response module operable to receive a query signal from a controller and to send a response signal in response to said query signal;
b) a cryogen supply; and
c) a cryogen control module which comprises
i) an inquiry mechanism operable to send an inquiry signal to said cryoprobe and to uniquely identify said cryoprobe upon receipt of a response signal sent by said cryoprobe in answer to said inquiry signal;
ii) a first memory for recording information about uniquely identified cryoprobes;
iii) a cryogen flow control mechanism for regulating flow of cryogen from said cryogen supply to said cryoprobe; and
iv) a first calculation module for calculating cryogen flow commands which influence operation of said cryogen flow control mechanism, said calculation being based at least in part on information associated with said uniquely identified cryoprobe and stored in said first memory.

2. The system of claim 1, wherein said response module comprises a second calculator operable to calculate said response signal as a mathematical function of a value presented by said inquiry signal.

3. The system of claim 1, wherein said response module is operable to recognize when a received inquiry code possesses a predetermined characteristic, and to emit a characteristic response when an inquiry code having said predetermined characteristic is recognized.

4. The system of claim 3, wherein said predetermined characteristic is a digital code uniquely associated with said cryoprobe.

5. The system of claim 1, wherein said inquiry signal is sent when an electronic communications pathway is first established between said controller and said cryoprobe.

6. The system of claim 1, further comprising
d) an information source physically distinct from said controller and from said cryoprobe, readable by said controller and comprising information characterizing said cryoprobe.

7. The system of claim 6, wherein said second memory device comprises a recordable magnetic strip.

8. The system of claim 6, wherein said second memory device comprises an optically readable code.

9. The system of claim 1, wherein said controller is programmed to record results of operational testing of said cryoprobe.

10. The system of claim 9, wherein said controller is operable to record information attesting to said cryoprobe having undergone operational testing, and to prevent clinical use of said cryoprobe if such information has not been so recorded.

11. The system of claim 1, wherein said controller is programmed to record events of usage of said cryoprobe, and to prevent supply of cryogen to said cryoprobe if more than a predetermined amount of usage has been recorded.

12. The system of claim 6, wherein said information characterizing said cryoprobe comprises manufacturing specifications describing said cryoprobe.

13. The system of claim 1, wherein said controller is operable to receive and record sensor values detected during testing of said cryoprobe, and is further operable to calculate cryogen supply parameters for use during operation of said cryoprobe as a function of said recorded values.

14. A method for cryosurgery, comprising
a) reading information descriptive of a cryoprobe into a controller;
b) using said controller to associate said read information with a cryoprobe by
i) sending an inquiry signal based on said read information to a cryoprobe,
ii) receiving a response signal from said cryoprobe in response to said inquiry signal; and
iii) associating said read information with said cryoprobe if and only if said response signal conforms to predetermined criteria; and
c) using said controller to calculate commands controlling supply of cryogen to said cryoprobe, said calculation being at least partially based on read information which said controller has associated with said cryoprobe in response to said response signal.

15. The method of claim 14, wherein said read information comprises a code which, when sent to said cryoprobe in an inquiry signal, will provoke a response signal which uniquely identifies said cryoprobe.

16. The method of claim 14, wherein said read information comprises at least one of a group consisting of
a) information characterizing a usage history of said cryoprobe;
b) data derived from an operational test of said cryoprobe;
c) a type designation for said cryoprobe; and
d) a descriptive characterization of said cryoprobe.

17. A method for regulating use of a cryoprobe, comprising:
a) reading information descriptive of a cryoprobe into a controller;
b) associating said read information with a cryoprobe attached to a controller by
i) sending an inquiry signal based on said read information to a cryoprobe,
ii) receiving a response signal from said cryoprobe in response to said inquiry signal; and
iii) associating said read information with said cryoprobe and with a unique identity tag if and only if said response signal conforms to predetermined criteria;
c) recording an activity history of said cryoprobe by recording probe usage events related to use of said cryoprobe in a memory record associated with said unique identity tag; and
c) regulating use of said cryoprobe by controlling cryogen flow as a function of recorded information associated with said unique identity tag.

18. A method of charging a customer for cryoprobe use, comprising:
a) supplying to a customer a plurality of cryoprobes, each associated with a unique identifying tag;
b) using a cryosurgery control module to record usage statistics for each of said cryoprobes when said cryoprobes are used; and
c) charging a customer according to said recorded usage statistics.

19. A cryotherapy system comprising
a) a cryoprobe which comprises
i) a treatment head coolable by delivery thereto of a cryogen; and
ii) an embedded electronic module which comprises a memory and a communication interface;
b) a cryogen supply; and
c) a cryogen control module operable to regulate flow of cryogen from said cryogen supply to said cryoprobe in response to information received from said embedded electronic module,
wherein at least one of a group consisting of said electronic module and said control module is programmed to record operational testing of said cryoprobe, and said control module is operable to prevent clinical use of said cryoprobe if said cryoprobe has not been operationally tested.

20. The system of claim 19, wherein at least one of a group consisting of said electronic module and said control module is programmed to record events of usage of said cryoprobe, and said control module is programmed to prevent supply of cryogen to said cryoprobe if more than a predetermined amount of usage has been recorded.

21. The system of claim 19, wherein operating values detected during testing of said cryoprobe are written into said memory of said electronic module and are useable by said control module during calculation of cryogen supply parameters used during operation of said cryoprobe.

22. A method for regulating use of a cryoprobe, comprising:
a) recording a unique identification code in a read-only memory embedded in a cryoprobe;
b) recording an activity history of said cryoprobe by recording probe usage events associated with said unique identification code; and
c) regulating use of said probe by identifying said cryoprobe by reading said unique identification code from said read-only memory, and choosing between supplying cryogen to said probe and denying supply of cryogen to said probe, said choice being determined algorithmically as a function of a recorded probe activity history identified by said unique identification code.

23. A method for cryosurgery, comprising:
a) recording, in an electronic module embedded in a cryoprobe, information characterizing said probe; and
b) algorithmically regulating use of said probe by choosing between supplying cryogen to said probe and denying supply of cryogen to said probe, said choice being determined algorithmically based on a reading of said recorded probe characterization and upon a recorded history of usage of said cryoprobe.

24. A method charging a customer for cryoprobe use, comprising:
a) supplying to a customer a plurality of cryoprobes each of which comprises an electronic module which comprises a read-only memory holding a unique identity number;
b) using a cryosurgery control module to record usage statistics for each of said cryoprobes when said cryoprobes are used; and
c) charging a customer according to said recorded usage statistics.
